(19) United States
(12) Patent Application Publication
(10) Pub. No.: US 2025/0205160 A1
(43) Pub. Date: Jun. 26, 2025

(54) STABLE PHARMACEUTICAL COMPOSITION OF AN AMINE DRUG

(71) Applicant: SUN PHARMACEUTICAL INDUSTRIES LIMITED, Mumbai (IN)

(72) Inventors: Kapil AGNIHOTRI, Gurugram (IN); Pawan Kumar SINGH, Gurugram (IN); Anuj Kumar FANDA, Gurugram (IN); Sumit MADAN, Gurugram (IN); Romi SINGH, Gurugram (IN); Avinash WAGH, Vadodara (IN); Daxesh PATEL, Vadodara (IN); Virendra Ramkrupal KURIL, Vadodara (IN)

(21) Appl. No.: 19/076,661

(22) Filed: Mar. 11, 2025

Related U.S. Application Data
(63) Continuation of application No. PCT/IB2023/062817, filed on Dec. 15, 2023.

(30) Foreign Application Priority Data
Dec. 17, 2022 (IN) 202221073257

(51) Int. Cl. A61K 9/16; A61K 9/48; A61K 31/381; A61K 47/18; A61K 47/22; A61K 47/38

(52) U.S. Cl. CPC A61K 9/167; A61K 9/4891; A61K 31/381; A61K 47/183; A61K 47/22; A61K 47/38

(57) ABSTRACT

The present disclosure relates to a stable multiparticulate composition comprising coated discrete units of an amine drug or a pharmaceutically acceptable salt thereof, with stabilizing amount of one or more nitrite quencher. The composition provides effective control of the level of nitrosamine drug substance-related impurities (NDSRIs) in the drug product during shelf life of the product.

### FIELD OF THE INVENTION

The present invention relates to a stable multiparticulate pharmaceutical composition comprising an amine drug or a pharmaceutically acceptable salt thereof and a stabilizing amount of at least one nitrite quencher. The composition shows an improved stability with controlled levels of potentially carcinogenic nitrosamine drug substance-related impurities (NDSRIs).

### BACKGROUND OF THE INVENTION

Lately there has been a spate of drug product withdrawal caused by nitrosamine drug substance-related impurities (NDSRIs). Such nitrosamine impurities can form during manufacturing or subsequent storage or during stability studies of the drug product. Such impurities may be formed due to use of variety of commonly utilized excipients or due to degradation of active pharmaceutical ingredients containing primary, secondary, tertiary or quaternary amine groups in their structures.

The term nitrosamine describes a class of compounds having the chemical structure of a nitroso group bonded to an amine (R1N(—R2)-N═O), as shown in Scheme 1. The compounds can form by a nitrosating reaction between amines (primary, secondary, tertiary, or quaternary amines) and nitrous acid (nitrite salts under acidic conditions).

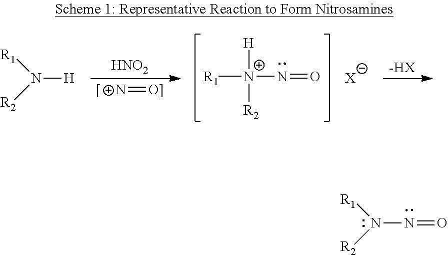

As per FDA guidance for industry for Control of Nitrosamine Impurities in Human Drugs, FDA has identified seven nitrosamine impurities that theoretically could be present in drug products: N-nitrosodimethylamine (NDMA), N-nitrosodiethylamine (NDEA), N-nitroso-N-methyl-4-aminobutanoic acid (NMBA), N-nitrosoisopropylethylamine (NIPEA), N-nitrosodiisopropylamine (NDIPA), N-nitrosodibutylamine (NDBA), and N-nitrosomethylphenylamine (NMPA). Five of them (NDMA, NDEA, NMBA, NIPEA, and NMPA) have actually been detected in drug substances or drug products. Such Nitrosamine compounds are potent genotoxic agents in several animal species and some are classified as probable or possible human carcinogens by the International Agency for Research on Cancer (IARC).

Global regulatory bodies including FDA and EMEA has set guidelines for mitigating the risk associated with nitrosamine impurities and outlined the various issues impacting on the drug product. FDA strongly urges applicants “to develop control strategies and/or design approaches to control nitrosamine drug substance-related impurities within acceptable levels.

Various references have talked about methods to optimize the level of nitrosamine impurities by using approaches. However, there are limited disclosures available which are actually enabled to control the nitrosamine impurities in drug product during storage period.

The inventors in the present application have evaluated various formulation strategies to mitigate the risk associated by nitrosamine compounds in drug product, and surprisingly arrived at the present invention.

### SUMMARY OF THE INVENTION

The present disclosure relates to a stable composition comprising coated discrete units of an amine drug or a pharmaceutically acceptable salt thereof, with stabilizing amount of one or more nitrite quencher. The composition provides effective control of the level of nitrosamine drug substance-related impurities (NDSRIs) in the drug product during shelf life of the product.

In one aspect the composition of the amine drug according to the present invention can be mixed with or sprinkled on food and swallowed intact with the food. The dosage forms have higher acid resistance as compared to existing extended or delayed release capsules or tablets but at the same time are bioequivalent. The multiparticulate composition of the present invention can be sprinkled and may further be suitable for administration via nasogastric tube (NG) or via other enteral feeding tube and has high acid resistance stability after passage through NG tube as well. However, it presents a challenge for the present inventors to formulate a stable multiparticulate sprinkle dosage form of an amine containing acid labile drug with controlled level of impurities including the nitrosamine drug substance-related impurities (NDSRIs).

In one aspect according to the present invention the composition is a multiparticulate composition.

In another aspect the present invention also provides method for controlling nitrosamine drug substance-related impurities (NDSRIs) in amine containing drug products.

In yet another aspect, the present invention provides a method for preparing a stable pharmaceutical composition of an amine drug comprising an amine drug or a pharmaceutically acceptable salt and at least one nitrite quencher, wherein the nitrite quencher is placed in the composition in a way to effectively control the formation of nitrosamine drug substance-related impurities (NDSRIs) in the composition, or such other impurities related to drugs with primary, secondary or tertiary amines.

### DESCRIPTION OF THE INVENTION

As used herein, the word “a” or “plurality” before a noun represents one or more of the particular nouns.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise.

The term “coating” as used herein the description, can be used interchangeably with the term “coat” or “layer” around the core.

The term “amine drug” as used herein the description, means a drug or its salt that has amine group present in the structure as primary, secondary, tertiary, or quaternary amines. The most commonly used amine drugs that can be used according to the present invention including but not limited to sartans, antidiabetic drugs, antihistamines, and antibiotics; for e.g. the drugs include Amoxicillin, Bendroflumethiazide, Betahistine, Bisoprolol, Bromazepam, Carvedilol, Cetirizine, Desloratadine, Diclofenac, Doxylamine, Duloxetine, Enalapril, Ergometrine, Felodipine, Flecainide, Fluoxetine, Hydrochlorothiazide (HCT), Levofloxacin, Metoprolol, Metformin, Mirabegron, Mirtazapine, Moxifloxacin, Nebivolol, Opipramol, Pergolide, Propranolol, Quetiapine, Ramipril, Roxithromycin, Sertraline, Sotalol, Sumatriptan, Sitagliptin, Valsartan, Varenicline, Metformin, Ranitidine, Gliclazide, Sitagliptin, Prucalopride, Clarithromycin, Oxcarbazepine, Sunitinib, Rivaroxaban, Citalopram, Escitalopram, Tamsulosin, Diltiazem, Amlodipine or pharmaceutically acceptable salt thereof, and combination thereof.

The term “about” as used herein, refers to any value that lies within the range defined by a variation of up to ±10% of the value.

The term “shelf life” as used in herein, means the shelf life of the drug product, in its form as a product sold for use by consumers, during which period the product is suitable for use by a patient. The shelf life of the drug product can be greater than 3, 6, 12, 18, or preferably 24 months. The shelf life may be achieved when the product is stored at room temperature at about 25° C.

The present invention provides a stable pharmaceutical composition of an amine drug or a pharmaceutically acceptable salt thereof, and at least one nitrite quencher in the composition, wherein the nitrite quencher effectively controls the level of the nitrosamine drug substance-related impurities (NDSRIs) in the drug products having primary, secondary or tertiary amine in their structure.

In another embodiment according to the present invention, the pharmaceutical composition is a stable multiparticulate composition comprising coated discrete units of an amine drug or a pharmaceutically acceptable salt thereof, with stabilizing amount of one or more nitrite quencher effective for controlling the level of nitrosamine related impurities in the drug product during shelf life of the product.

In a further embodiment according to the present invention, the nitrite quencher is present in the composition in such a way that it effectively controls the level of nitrosamine drug substance-related impurities (NDSRIs) in the drug product.

In some embodiments according to the present invention, the nitrite quencher is present in the core of the composition within a modified release coating for effective control of the level of nitrosamine drug substance-related impurity in the drug product.

In some embodiments according to the present invention, the nitrite quencher is present in the composition within a modified release coating for effective control of the level of nitrosamine drug substance-related impurity in the drug product.

The term “dosage form” as used herein the description, can be used interchangeably with the term ‘composition’ or ‘formulation’ or ‘pharmaceutical preparation’ or ‘drug product’, wherein the drug product is defined as a composition of amine drug and one or more excipients.

The nitrosamine drug substance-related impurities (NDSRIs) in the composition of drug product may be formed due to nitrosating reaction between amines (primary, secondary, tertiary, or quaternary amines) of drug and/or excipients present in the drug product, and the applicant surprisingly found that the nitrite quencher in claimed concentration ranges and arrangement play crucial role in controlling nitrosamine drug substance-related impurities (NDSRIs) as well as other impurities simultaneously in the composition below acceptable levels.

In some embodiment according to present invention, the stable multiparticulate composition is a sprinkle dosage form of amine drug and stabilizing amount of at least one nitrite quencher. In one aspect according to above embodiment, the nitrite quencher characteristically controls or prevents formation of nitrosamine drug substance-related impurity in the drug product upon storage or acid exposure.

In one embodiment, the stable multi-particulate sprinkle composition of the invention may be modulated to provide a modified release such as a delayed release or an extended release of the drug, or a combination of delayed and extended drug release.

The term “stable” as used herein, refers to a chemical stability of the composition which means that the level of nitrosamine drug substance related impurity (NDSRIs) in the composition remains below about 1 ppm, preferably below about 0.99 ppm, below about 0.98 ppm, below about 0.97 ppm, below about 0.96 ppm, below about 0.95 ppm, below about 0.94 ppm, below about 0.93 ppm, below about 0.92 ppm, below about 0.91 ppm, below about 0.90 ppm, below about 0.89 ppm, below about 0.88 ppm, below about 0.87 ppm, below about 0.86 ppm, below about 0.85 ppm, below about 0.84 ppm, below about 0.83 ppm, below about 0.82 ppm, below about 0.81 ppm or below about 0.80 ppm, upon storage under the temperature and humidity conditions of 40° C./75% RH for at least 6 months and at controlled room temperature condition of 25° C./60% RH for at least 12 months, 18 months or 24 months.

The term “stable” as used herein, also refers to physical stability which means that the modified release coat over the multiparticulate composition retain its structural integrity and does not rupture in a significant way after exposure to acidic environment for the given time period as determined by the drug release and also includes chemically stability which means the multiparticulate composition is stable when stored under the temperature and humidity conditions of 40° C./75% RH and 30° C./65% RH for at least 6 months or at controlled room temperature condition of 25° C./60% RH for at least 12 months, 18 months or 24 months

According to another embodiment, the disclosed stable multiparticulate composition comprising coated discrete units of an amine drug or a pharmaceutically acceptable salt thereof, with one or more nitrite quencher in an amount effective for controlling the level of nitrosamine drug substance-related impurities (NDSRIs) in the drug product is coated with a modified release coating. In one aspect of the above embodiment, the modified release coating is a delayed release coating. In another aspect of the above embodiment, the modified release coating is an extended release coating. In yet another aspect of the above embodiment the modified release coating is a combination of delayed and extended release coating.

In some embodiment according to the present invention the disclosed stable multiparticulate composition comprising coated discrete units of an amine drug or a pharmaceutically acceptable salt thereof, with one or more nitrite quencher in an amount effective for controlling the level of nitrosamine drug substance-related impurities (NDSRIs) in the drug product is coated with a coating comprising at least one enteric release polymer. In one aspect of the above embodiment, the enteric polymer is a pH sensitive enteric release polymer.

In another embodiment according to present invention, the disclosed stable multiparticulate dosage form comprises an amine drug or a pharmaceutically acceptable salt thereof, wherein the amine drug has a primary, secondary, tertiary or quaternary amine group in the structure.

In another embodiment according to present invention, the disclosed stable multiparticulate dosage form comprises duloxetine or a pharmaceutically acceptable salt thereof with one or more nitrite quencher for controlling the level of nitrosamine drug substance related impurities (NDSRIs) to a level below about 1 PPM. In one aspect the level of nitrosamine drug substance related impurity (NDSRIs) in the composition remains below about 1 ppm, preferably below about 0.99 ppm, below about 0.98 ppm, below about 0.97 ppm, below about 0.96 ppm, below about 0.95 ppm, below about 0.94 ppm, below about 0.93 ppm, below about 0.92 ppm, below about 0.91 ppm, below about 0.90 ppm, below about 0.89 ppm, below about 0.88 ppm, below about 0.87 ppm, below about 0.86 ppm, below about 0.85 ppm, below about 0.84 ppm, below about 0.83 ppm, below about 0.82 ppm, below about 0.81 ppm or below about 0.80 ppm upon storage at stored under the temperature and humidity conditions of 40° C./75% RH for at least 6 months and at controlled room temperature condition of 25° C./60% RH for at least 12 months, 18 months or 24 months. In one aspect of the above embodiment the nitrosamine drug substance related impurities (NDSRIs) is nitrosoduloxetine. In one aspect, the level of nitrosoduloxetine is below about 0.83 PPM for shelf life period.

The term “nitrosoduloxetine” as used herein the specification, refers to the nitrosamine drug substance related impurities (NDSRIs) associated with duloxetine or its pharmaceutical acceptable salts. Such impurities may be present in active pharmaceutical ingredients or can be formed in the drug product up on storage.

In yet another embodiment according to present invention the disclosed stable multiparticulate dosage form comprises an amine drug or a pharmaceutically acceptable salt and at least one nitrite quencher selected from ascorbic acid or salt thereof; amino acids selected from cysteine or salt thereof, histidine or salt thereof, glycine or salt thereof, arginine or salt thereof. In one aspect, the said nitrite quencher may further be combined with additional components selected from saccharin, meglumine, alkalizer, or combination thereof.

According to another embodiment of the present invention, the stable multiparticulate dosage form comprising duloxetine or a pharmaceutically acceptable salt thereof with at least one nitrite quencher is a sprinkle dosage form and wherein the nitrite quencher characteristically controls or prevents formation of nitrosamine drug substance-related impurities in the drug product. In one aspect of the above embodiment, the stable composition of duloxetine or a pharmaceutically acceptable salt thereof has higher acid resistance as compared to existing duloxetine delayed release capsules. In yet another aspect, the multiparticulate sprinkle dosage form is bioequivalent to the existing duloxetine delayed release capsules that have an enteric layer of hydroxypropyl methylcellulose acetate succinate.

In yet another embodiment the present invention provides a stable multiparticulate sprinkle dosage form which when administered after sprinkling on soft food has enhanced oral bioavailability as compared to the marketed duloxetine delayed release capsules.

According to another aspect of the present invention, the stable multiparticulate sprinkle dosage form is present in the form of tablets, capsules, sachet or granules.

The term “granules”, “pellets” or “spheroids” as used herein can be used interchangeably, and includes agglomeration from apparent solid powder particles to large multi-particulates. The agglomeration may be achieved by either granulation, compaction, extrusion, slugging, drug loading or the like. Such granules, pellets or spheroids have good flow property and these may be spherical or oval in shape, and may have a density higher than a powder. The granules or pellets or spheroids are coated, in particular, they can be preferably coated with at least one functional coating, as described herein. The average diameter of coated pellets or granules is about 500 μm to about 1800 μm, or about 600 μm to about 1500 μm. Also included are granules as defined in USP <1151>, which is incorporated herein by reference.

In yet another embodiment, the stable multiparticulate sprinkle dosage form according to the present invention comprises discrete units (core subunits) coated with an enteric polymer.

An enteric or delayed release coating layer according to present invention comprises an enteric polymer and one or more pharmaceutically acceptable excipients. Suitable enteric polymers are selected from the group comprising of methacrylic acid copolymers, cellulose acetate phthalate, cellulose acetate succinate, polymethacrylic acid/acrylic acid copolymer, Kollicoat MAE 30DP, Eudragit, hydroxypropyl methylcellulose phthalate, polyvinyl acetate phthalate, hydroxyethyl ethyl cellulose phthalate, cellulose acetate tetrahydrophthalate, acrylic resin, and mixtures thereof. The enteric polymer is present in an amount of more than 60%, or more than 70%, or more than 75% based on the total weight of the enteric coating layer, e.g. about 55% to about 98% by weight hydroxypropyl methylcellulose phthalate based on the total weight of the enteric coating layer. The enteric layer is present in an amount of at least about 15%, or at least about 35% by weight based on the weight of the core subunit without the enteric coat. Preferably, the enteric layer is present in an amount of about 15 to about 30%, or in an amount of about 17% to about 25% by weight based on the weight of the core subunit without the enteric coat. More preferably, the enteric layer is present in an amount of about 21%, about 23%, about 25% by weight based on the weight of the core subunit without the enteric coat. The thickness of enteric coating layer is between 30 μm to 50 km.

In a further embodiment of the present invention, the discrete unit or the multiparticulate is present in the form of pellets, beads, particles, granules, or mini-tablets. In one aspect, the discrete unit comprises inert cores coated with the drug layer. In yet another aspect, the discrete unit comprises inert cores coated with the drug layer containing at least one nitrite quencher. In a further aspect, the discrete unit comprises inert cores coated with the drug layer containing a combination of two or more nitrite quenchers.

In one embodiment according to the present invention, the stable multiparticulate dosage form comprising duloxetine or a pharmaceutically acceptable salt thereof with at least one or more stabilizer selected from ascorbic acid, tocopherol, butylated hydroxyanisole, butylated hydroxytoluene, propyl gallate, sodium sulfites selected from sodium sulfite, sodium bisulfite, sodium metabisulfite, amines or amino acids like cysteine, acetyl cysteine, histidine, glycine, arginine or a combination thereof. In another embodiment, the multiparticulate are discrete units comprising an inert core coated with the drug layer containing a stabilizer, amino acid or an alkalizer. In one aspect, the stabilizer is a single component. In another aspect, the stabilizer in the composition of present invention is a combination component and it can be a combination of two or more stabilizer or in combination with nitrite quencher in a ratio effective in controlling nitrosamine drug substance-related impurities.

In another embodiment according to the present invention, the stable multiparticulate dosage form comprising duloxetine or a pharmaceutically acceptable salt thereof with at least one or more alkalizer, wherein alkalizer is selected from magnesium oxide, meglumine, aluminum oxide, ammonium hydroxide, magaldrate, an alkali metal salt or alkaline earth metal salt, such as sodium bicarbonate, calcium carbonate or sodium citrate, an alkali metal hydroxide such as sodium hydroxide, potassium hydroxide or lithium hydroxide, or an alkaline earth metal hydroxide such as calcium hydroxide or magnesium hydroxide, with magnesium oxide or meglumine being preferred. In some aspects of the embodiment, the alkalizer is used with a nitrite quencher.

In yet another embodiment of the present invention, the multiparticulate sprinkle dosage form contains duloxetine or pharmaceutically acceptable salts thereof in an amount of not less than 10%, preferably not less than about 15%, more preferably in an amount of about 18-25% by weight of the dosage form.

According to another embodiment, the enteric polymer is present in an amount of not less than about 65% by weight based on the weight of the enteric coating.

According to another embodiment, the enteric layer is present in an amount of about 15% to about 30% by weight based on the weight of the core subunit without the enteric coat.

According to another embodiment, the enteric layer is present in an amount of about 18% to about 25% by weight based on the weight of the core subunit without the enteric coat.

According to another embodiment, the multiparticulate sprinkle dosage form comprises a cushioning agent.

According to another embodiment of this aspect, the cushioning agent is present in an amount of not less than 2% and not more than 20% based on the weight of the enteric-coated pellet.

According to another embodiment of this aspect, the multiparticulate sprinkle dosage form is resistant to alcohol induced dose dumping.

According to another embodiment of this aspect, the multiparticulate sprinkle dosage form can be placed in contact with soft food before administration without affecting the stability of the enteric coating.

According to another embodiment of this aspect, the multiparticulate sprinkle dosage form can be placed in contact with soft food before administration without affecting the levels of nitrosamine compound related impurities.

According to another embodiment of this aspect, the multiparticulates when sprinkled on soft food are stable for at least 60 minutes.

According to one embodiment of this aspect, the multiparticulates when sprinkled on soft food having pH less than or equal to 5 are stable for about 90 minutes.

According to another embodiment of this aspect, the multiparticulates when sprinkled on soft food having pH more than 5 at e.g., 25° C. or 37° C. are stable for about 60 minutes.

According to another embodiment of this aspect, the multiparticulate sprinkle dosage form is suitable for administration in intact form. According to one embodiment of this aspect, the multiparticulate sprinkle dosage form is suitable for administration to a patient via a feeding tube. According to another embodiment of this aspect, the multiparticulate sprinkle dosage form has higher acid resistance stability after recovery through a feeding tube.

Discrete units may be in a form selected from the group consisting of pellets, beads, particles, granules, and mini-tablets and the like.

Provided herein, for example, is a multiparticulate sprinkle dosage form comprising a plurality of pellets each comprising: a drug layered pellet comprising an inert core, and a drug layer surrounding the inert core comprising duloxetine or a pharmaceutically acceptable salt thereof, with stabilizing amount of at least one nitrite quencher, an enteric coating surrounding the drug layered subunit, wherein the enteric coating comprises an enteric polymer; and a finishing layer.

Provided herein, for example, is a multiparticulate sprinkle dosage form comprising a plurality of pellets each comprising: a drug layered pellet comprising an inert core, and a drug layer surrounding the inert core comprising duloxetine or a pharmaceutically acceptable salt thereof, with stabilizing amount of at least one nitrite quencher, a seal coating, an enteric coating surrounding the drug layered subunit, wherein the enteric coating comprises an enteric polymer; and a finishing layer.

In some embodiments, the multiparticulate dosage form has nitrosamine drug substance related impurity level below about 1 ppm, preferably below about 0.9 ppm, more preferably below about 0.83 ppm. In yet another aspect, the multiparticulate dosage form has less than 0.2% w/w of 1-naphthol impurity upon storage at 40° C./75% RH for at least 6 months or at 25° C./60% RH for about 12 months or about 24 months. For example, in some embodiments, the enteric polymer of disclosed discrete units is hydroxypropyl methylcellulose phthalate and the finishing layer comprises polyethylene glycol 6000. In some embodiments, the enteric polymer is methacrylic acid and ethyl acrylate copolymer dispersion.

Provided herein, for example, is a multiparticulate sprinkle dosage form to a patient, comprising a plurality of discrete units, each discrete unit comprising:
a core subunit comprising:
   a drug layered pellet comprising an inert sphere coated with a drug layer, wherein the drug layer comprises duloxetine hydrochloride and at least on nitrite quencher, and
   a sub coating comprising hydroxypropyl methylcellulose (HPMC/Hypromellose), wherein the sub coating covers the drug-layered pellet;
   an enteric coating covering the sub coating; and a
   a finishing.

In another example, the a multiparticulate sprinkle dosage form is provided to a patient, comprising a plurality of discrete units, each unit comprising:
a core subunit comprising:
   a drug layered pellet comprising an inert sphere coated with a drug layer, wherein the drug layer comprises duloxetine hydrochloride and at least on nitrite quencher, and
   a seal coating comprising hydroxypropyl methylcellulose (HPMC/Hypromellose), wherein the seal coating covers the drug layered pellet; and
   an enteric coating covering the seal coating; and
   optionally, a stabilizer provided with the nitrite quencher.

In one embodiment the present invention provides a stable multiparticulate sprinkle dosage form comprising duloxetine or a pharmaceutically acceptable salt thereof, wherein the dosage form comprises multiparticulate coated with an enteric coating followed by finishing layer comprising cushioning agent.

According to one embodiment of this aspect, the multiparticulate sprinkle dosage form comprises: (a) an inert core, (b) optionally an inner seal coat, (c) a drug layer, (d) optionally a sub coating layer, (c) an enteric coating layer, (f) optionally a finishing layer. According to another embodiment of this aspect, the cushioning agent is present in the finishing layer. According to another embodiment of this aspect, the cushioning agent is present in an amount of not less than 2% and not more than 20% based on the total weight of the dosage form.

According to another embodiment of this aspect, the multiparticulate sprinkle dosage form has higher acid resistance as compared to the marketed duloxetine delayed release capsules.

Another aspect of the present invention provides a multiparticulate sprinkle dosage form comprising duloxetine or a pharmaceutically acceptable salt thereof, wherein the dosage form can be placed in contact with soft food before administration for at least 60 minutes without affecting the stability of the enteric coating.

Yet another aspect of the present invention provides a multiparticulate sprinkle dosage form comprising duloxetine or a pharmaceutically acceptable salt thereof, suitable for administration to a patient via enteral feeding tube such as NG tube or G tube, and has high acid resistance as compared to the marketed duloxetine delayed release capsules.

According to one embodiment of this aspect, the multiparticulates have higher acid resistance stability after recovery through a combination of a syringe and the feeding tube.

The term “duloxetine”, as used herein, refers to duloxetine and pharmaceutically acceptable salts thereof, including hydrates and solvates thereof, and crystalline or amorphous forms thereof. The preferred form is the hydrochloride salt.

The term “multiparticulate sprinkle dosage form” or “formulation” or “composition”, as used herein, refer to plurality of physically discrete units, wherein each unit contains a predetermined quantity of active ingredient in association with pharmaceutically acceptable excipients. The dosage form used herein may be selected from conventional hard capsule (e.g. a capsule of gelatin, HPMC or a starch derivative), an easy to open capsule, or sachet. The multiparticulate sprinkle dosage form can provide dosage of about 10 mg, 20 mg, 30 mg, 40 mg, 50 mg, 60 mg, 70 mg, 80 mg, 90 mg, 100 mg, 110 mg, or 120 mg per dose or per day.

The term “multiparticulate” as used herein refers to plurality of physically discrete units, wherein each discrete unit is coated with an enteric polymer. The discrete unit comprises a drug core in the form of pellets, beads, particles, granules, or mini-tablets. In the present specification, the term “multiparticulates”, “discrete units”, “pellets”, “beads”, are used interchangeably.

The term “pharmaceutically acceptable excipients”, as used herein, includes excipients that may be added in the stable multiparticulate sprinkle dosage form.

The term “cushioning agent” as used herein encompasses inert substances that provide flexibility to cores coated with enteric polymer such that when exposed to acidic environment for prolonged period, the enteric coat substantially retain their structural integrity and do not rupture in a significant way. Also, the cushioning agent protects the enteric coat from damage due to accidental crushing or chewing of the pellets in the mouth.

The multiparticulate sprinkle dosage form comprises discrete units coated with an enteric polymer. The discrete units are drug cores covered with an enteric polymer. The drug core may comprise an inert core coated with a drug layer comprising drug and one or more pharmaceutically acceptable excipients. Alternatively, the drug core comprises the drug and one or more pharmaceutically acceptable excipients which are uniformly mixed. The inert core comprises sugar spheres or pellets of microcrystalline cellulose. The drug i.e. duloxetine hydrochloride is present in an amount of not less than about 10%, not less than about 15%, not less than about 24%, based on the total weight of the dosage form. Preferably, the drug i.e. duloxetine hydrochloride is present in an amount of about 24%, based on the total weight of the dosage form.

The term “weight of the dosage form” is construed to include only the fill weight i.e. excluding the capsule shell weight.

The particle size of the active defined as D₉₀ is not more than 50 μm, preferably not more than 20 μm. For example, a multiparticulate sprinkle dosage form comprises: (a) an inert core, (b) a drug layer, and (c) an enteric coating layer over the drug coated core. A seal coat may optionally be present between the inert core and the drug layer (inner seal coat), between the drug layer and the enteric layer (sub coat), or above the enteric layer (finishing layer). A nitrite quencher may be positioned suitably in a stabilizing amount for effectively controlling nitrosamine drug substance-related impurities.

The multiparticulate sprinkle dosage form may be present in the form of easy to open capsules, conventional hard capsules (e.g. a capsule of gelatin, HPMC or a starch derivative), or a sachet.

The multiparticulate sprinkle dosage form comprises one or more pharmaceutically acceptable excipients selected from one or more of fillers, binders, disintegrants, lubricants, glidants, anti-adherents, cushioning agents, and mixtures thereof.

Suitable diluents are selected from the group consisting of lactose, microcrystalline cellulose, starch, pregelatinized starch, calcium sulphate, calcium carbonate, powdered cellulose, mannitol, sorbitol, xylitol, lactitol, magnesium carbonate, dicalcium phosphate, tricalcium phosphate, calcium sulphate; and mixtures thereof.

Suitable binders are selected from the group consisting of cellulose derivatives (for example methylcellulose, hydroxypropyl cellulose, hydroxypropyl methylcellulose (HPMC/Hypromellose), ethylcellulose, hydroxyl ethyl cellulose, L-hydroxy propyl cellulose); polyvinylpyrrolidone (for example povidone, copovidone); starch (for example cornstarch, pre-gelatinized starch and hydroxypropyl starch); polymethacrylates (for example Eudragit RS, RL); and mixtures thereof.

Suitable disintegrants are selected from the group consisting of low substituted hydroxypropyl cellulose, crospovidone, crosscarmellose sodium, starch derivatives; and mixtures thereof.

Suitable lubricants are selected from the group consisting of magnesium stearate, calcium stearate, zinc stearate; stearic acid, hydrogenated vegetable oil, hydrogenated castor oil, glyceryl palmitostearate, glyceryl behenate, polyethylene glycols, corn starch, sodium stearyl fumarate, sodium benzoate, mineral oil, talc, and mixtures thereof.

Suitable glidants or antiadherents are selected from the group consisting of talc, colloidal silicon dioxide, magnesium trisilicate, powdered cellulose, starch, tribasic calcium phosphate; and mixtures thereof.

Suitable cushioning agents are selected from the group comprising polyethylene glycols, polyoxyethylenes, colloidal and/or amorphous silicon dioxide, microcrystalline cellulose, polyvinyl acetate, waxes, fats, lipids, gums, and mixtures thereof. Examples of waxes include carnauba wax, bees wax, sperm whale wax, etc. Examples of fats and lipids include lecithin, hydrogenated vegetable oils, hydrogenated castor oil, hydrogenated sesame oil, etc., Examples of gums include gum arabica, xanthan gum, gum acacia, etc. It was surprisingly found that the use of cushioning agent in the dosage form imparts mechanical stability to the dosage form and aids in increase of the acid resistance of the dosage form. The cushioning agents may be layered over the drug cores or the enteric coat or the seal coat; or may be present in the drug cores or in the enteric coat or in the seal coat. Preferably, the cushioning agent is present in the outer finishing layer in an amount of not less than 20% and not more than 80% based on the weight of the coating layer. The cushioning agent is present in an amount of not less than 2% and not more than 25%, not more than 20%, not more than 15%, not more than 10% based on the total weight of the dosage form. Preferably, the cushioning agent is present in an amount of not less than 2% and not more than 25%, more preferably in an amount of about 5% based on the total weight of the dosage form.

The enteric coating layer comprises an enteric polymer and one or more pharmaceutically acceptable excipients. Suitable enteric polymers are selected from the group comprising of methacrylic acid copolymers, cellulose acetate phthalate, cellulose acetate succinate, polymethacrylic acid/acrylic acid copolymer, hydroxypropyl methylcellulose phthalate, polyvinyl acetate phthalate, hydroxyethyl ethyl cellulose phthalate, cellulose acetate tetrahydrophthalate, acrylic resin, and mixtures thereof.

The seal coating layer is optional. The seal coating layer comprises a film forming polymer and one or more pharmaceutically acceptable excipients. Additional excipients present in the coating include one or more of film forming polymers, plasticizers, anti-adherents, opacifiers, colorants, pigments, antifoaming agents, and polishing agents.

Suitable film-forming polymers are selected from the group consisting of hydroxypropyl methylcellulose, ethyl cellulose, methylcellulose, hydroxyethyl cellulose, hydroxypropyl cellulose, sodium carboxymethyl cellulose, cellulose acetate, hydroxypropyl methylcellulose phthalate, cellulose acetate trimellitate, and methacrylic acid copolymers, e.g., Eudragit®, polyvinylpyrrolidone, polyvinylalcohol, polyethylene glycol, and mixtures thereof. Other suitable film-forming polymers which are known in the art may also be used. Many suitable film-coating products which are commercially available, e.g., Opadry® and Opaglos® may be used.

Suitable plasticizers are selected from the group consisting of propylene glycol, triethyl citrate, tributyl citrate, dibutyl sebacate, acetyl tributyl citrate, glyceryl monostearate, triacetin, polyethylene glycol, diethyl phthalate, acetylated monoglycerides, diacetylated monoglycerides, cetyl alcohol, and mixtures thereof.

Suitable anti-adherents are selected from the group consisting of talc, magnesium stearate, fumed silica, and mixtures thereof.

Suitable opacifiers are selected from the group consisting of titanium dioxide, manganese dioxide, iron oxide, silicon dioxide, and mixtures thereof.

Suitable coloring agents are selected from FDA approved colorants such as iron oxide, lake of tartrazine, allura red, titanium dioxide, and mixtures thereof.

Suitable polishing agents are selected from the group consisting of polyethylene glycols of various molecular weights or mixtures thereof, talc, surfactants (glycerol monostearate and poloxamers), fatty alcohols (stearyl alcohol, cetyl alcohol, lauryl alcohol and myristyl alcohol), waxes (carnauba wax, candelilla wax and white wax), and mixtures thereof.

Various solvents that may be employed during the preparation of the dosage form of the present invention are selected from the group consisting of water, methyl alcohol, ethyl alcohol, isopropyl alcohol, n-butyl alcohol, acetone, acetonitrile, chloroform, methylene chloride, water, and mixtures thereof.

The coating may be carried out by using any conventional coating techniques known in the art, such as spray coating in a conventional coating pan or fluidized bed processor, or dip coating.

The multiparticulate sprinkle dosage form is suitable for administration to pediatrics, geriatrics, patients with swallowing difficulties, i.e., dysphagic patients as well as for patients with feeding tubes in place. The multiparticulate sprinkle dosage forms are suitable for administration as sprinkle dosage form as well as via enteral feeding tubes such as NG tube and G tube. The dosage form is also suitable for administration as intact capsules.

The present invention covers method of administering duloxetine to a patient, comprising (a) providing a multiparticulate sprinkle dosage form in the form of discrete units coated with an enteric polymer (b) sprinkling the discrete units on soft food, and (c) administering the soft food orally.

The size of discrete units suitable to be sprinkled on soft food is less than about 2 mm, preferably less than about 1.5 mm.

The multiparticulates can be placed in contact with soft food of varying pH prior to administration. In case the food has acidic pH and the multiparticulates after placing in contact with food are not administered immediately and have a hold time before administration, the multiparticulates are then exposed to acidic environment in-vitro for additional time period in addition to exposure to acidic environment in stomach. Since, the sprinkle dosage form may undergo prolonged exposure to acidic environment in-vitro, hence they should have higher acid resistance as compared to the duloxetine delayed release formulations which are not suitable for administration with food. The multiparticulate sprinkle dosage form has higher acid resistance upon exposure to soft food as compared to the marketed duloxetine delayed release capsules.

The multiparticulate sprinkle dosage forms are also suitable for administration to a patient via enteral feeding tubes such as NG-tube or G-tube to patients who cannot safely swallow or are unable to take medication orally. The enteral feeding tube may be a NG-tube 3.5-16 French or a G-tube of 12-28 French. Successful delivery of sprinkle drug products through an enteral feeding tube requires that all of the beads (uncrushed) be able to safely pass through the feeding tube and not cause tube occlusions.

The multiparticulate sprinkle dosage forms can be suspended in a suitable vehicle, preferably water and administered via enteral feeding tube. For patients who have a feeding tube in place, duloxetine multiparticulate dosage form is administered by a method comprising: (a) providing a multiparticulate sprinkle dosage form in the form of discrete units coated with an enteric polymer, (b) dispersing the discrete units in a recommended volume of liquid medium placed in a syringe, (c) attaching the syringe to the feeding tube and delivering the contents through the feeding tube into the stomach, (d) flushing the feeding tube with an additional liquid medium to clear the tube.

The multiparticulate sprinkle dosage forms are suitable for administration through a NG-tube or G-tube of at least 12 French. The multiparticulates do not adhere on the walls of the tube. The enteric coat of the discrete units remains stable after passage through the enteral feeding tube. The multiparticulate dosage forms have higher acid resistance stability as compared to the marketed duloxetine delayed release capsules after recovery through a combination of syringe and the feeding tube.

The multiparticulate sprinkle dosage form can be administered orally at a low dose and may provide equivalent efficacy in comparison to the marketed duloxetine delayed release formulation. The low dose is at least 5%, at least 10%, at least 15%, at least 20%, at least 25%, at least 30%, at least 35%, or at least 40% lower than currently approved dose of marketed delayed release capsules.

The multiparticulate sprinkle dosage form may be used for the treatment of disease or condition requiring duloxetine therapy.

Some of the exemplary embodiment of the present invention are enumerated herein below:

In one embodiment, the present invention provides a pharmaceutical composition of amine drug comprising an amine drug and a stabilizing amount of at least one nitrite quencher, wherein the nitrite quencher is effectively controlling the nitrosamine drug substance-related impurities (NDSRIs) in the composition.

In one related aspect of the above embodiment in the pharmaceutical composition the nitrite quencher is selected from ascorbic acid or salt thereof, amino acids selected from cysteine or salt thereof, histidine or salt thereof, glycine or salt thereof, arginine or salt thereof. In another aspect the said nitrite quencher may further be combined with additional components selected from saccharin, meglumine, alkalizer or combination thereof. In yet another aspect the composition may further comprise butylated hydroxy anisole.

In yet another embodiment according to present invention the disclosed stable multiparticulate dosage form comprises an amine drug or a pharmaceutically acceptable salt and at least one nitrite quencher selected from ascorbic acid or salt thereof; amino acids selected from cysteine or salt thereof, histidine or salt thereof, glycine or salt thereof, arginine or salt thereof. In yet another aspect the composition further comprises a stabilizer selected from butylated hydroxy anisole.

In another related aspect of the above embodiment in the pharmaceutical composition the nitrite quencher is present in the composition in an amount of about 0.01% w/w to about 10% w/w of an input amine drug in the composition, preferably in an amount of about 0.1% w/w to about 5% w/w, more preferably in an amount of about 0.1% w/w to about 2% w/w.

In yet another related aspect of the above embodiment in the pharmaceutical composition the nitrite quencher is ascorbic acid. In one aspect the ascorbic acid is present in the composition in a concentration range of about 0.01% w/w to about 10% w/w of an input amine drug in the composition, preferably in an amount of about 0.1% w/w to about 5% w/w, more preferably in an amount of about 0.1% w/w to about 2% w/w.

In yet another related aspect of the above embodiment in the pharmaceutical composition the amine drug is selected from a compound with a primary, secondary, tertiary or quaternary amine group or a pharmaceutically acceptable salt thereof.

In some aspect of the above embodiment in the pharmaceutical composition the amine drug is selected from Amoxicillin, Bendroflumethiazide, Betahistine, Bisoprolol, Bromazepam, Carvedilol, Cetirizine, Desloratadine, Diclofenac, Doxylamine, Duloxetine, Enalapril, Ergometrine, Felodipine, Flecainide, Fluoxetine, Hydrochlorothiazide (HCT), Levofloxacin, Metoprolol, Metformin, Mirabegron, Mirtazapine, Moxifloxacin, Nebivolol, Opipramol, Pergolide, Propranolol, Quetiapine, Ramipril, Roxithromycin, Sertraline, Sotalol, Sumatriptan, Sitagliptin, Valsartan, Varenicline, Metformin, Ranitidine, Gliclazide, Sitagliptin, Prucalopride, Clarithromycin, Oxcarbazepine, Sunitinib, Rivaroxaban, Citalopram, Escitalopram, Tamsulosin, Diltiazem, Amlodipine or pharmaceutically acceptable salt thereof, and combination thereof.

In yet another related aspect of the above embodiment in the pharmaceutical composition the amine drug is duloxetine or a pharmaceutically acceptable salt thereof.

In a further related aspect of the above embodiment in the pharmaceutical composition, the nitrite quencher and the amine drug are present in the composition within a modified release coating. In one related aspect, the modified release coating is an enteric coating or a delayed release coating.

In one aspect of the embodiment in the pharmaceutical composition, the modified release coating comprises an enteric polymer. In another aspect in the pharmaceutical composition, the enteric polymer is a pH sensitive polymer.

In some aspects of the above embodiment in the pharmaceutical the enteric polymer is selected from methacrylic acid copolymers, cellulose acetate phthalate, cellulose acetate succinate, polymethacrylic acid/acrylic acid copolymer, Kollicoat MAE 30DP, Eudragit, hydroxypropyl methylcellulose phthalate, polyvinyl acetate phthalate, hydroxyethyl ethyl cellulose phthalate, cellulose acetate tetrahydrophthalate, acrylic resin, or mixtures thereof.

In some aspects of the above embodiments, the pharmaceutical composition is a multiparticulate composition, comprising of plurality of discrete units.

In some aspects of the above embodiments, the pharmaceutical composition is in the form of a sachet, pouch or capsule.

In some related aspects of the above embodiments in the pharmaceutical the nitrosamine drug substance-related impurities (NDSRIs) is nitrosoduloxetine.

In one related aspect of the above embodiments, the pharmaceutical composition comprises discrete unit which is present in form of granules, pellets, beads, particulates, spheroids or minitablets.

In some aspects of the above embodiment in the pharmaceutical composition, the amine drug is layered on a core with at least one nitrite quencher.

In one aspect of the above embodiment, the pharmaceutical composition is further coated with a protective layer or seal coating. In an optional aspect, the composition is further surrounded by a finishing layer.

In one aspect of the above embodiment, the pharmaceutical composition is further packed with an oxygen absorber or oxygen scavenger.

In some aspect of the above embodiment in the pharmaceutical composition the nitrosamine drug substance-related impurities (NDSRIs) is controlled to a level below about 1 ppm. In one aspect of the related embodiment the nitrosamine drug substance related impurity is controlled to a level below about 0.99 ppm, below about 0.98 ppm, below about 0.97 ppm, below about 0.96 ppm, below about 0.95 ppm, below about 0.94 ppm, below about 0.93 ppm, below about 0.92 ppm, below about 0.91 ppm, below about 0.90 ppm, below about 0.89 ppm, below about 0.88 ppm, below about 0.87 ppm, below about 0.86 ppm, below about 0.85 ppm, below about 0.84 ppm, below about 0.83 ppm, below about 0.82 ppm, below about 0.81 ppm or below about 0.80 ppm upon storage at 40° C./75% RH for at least 6 months and at controlled room temperature condition of 25° C./60% RH for at least 12 months, 18 months or 24 months.

In one embodiment the present invention provides a pharmaceutical composition of duloxetine comprising duloxetine or a pharmaceutical acceptable salt thereof, and a stabilizing amount of at least one nitrite quencher, wherein the composition is characterized by nitrosamine drug substance-related impurities (NDSRIs) in the composition below about 1 ppm, preferably below about 0.9 ppm, more preferably below about 0.83 ppm

In one aspect according to above embodiment, in the pharmaceutical composition the duloxetine or a pharmaceutical acceptable salt thereof, and a stabilizing amount of at least one nitrite quencher for effectively controlling nitrosamine drug substance-related impurities.

In an aspect, the nitrite quencher is present in the composition within a modified release coating. In another aspect according to the above embodiment, in the pharmaceutical composition the modified release coating is an enteric coating or a delayed release coating.

In some aspect according to the above embodiment, in the pharmaceutical composition the modified release coating comprises an enteric polymer. In one aspect according to present embodiment, in the pharmaceutical composition the enteric polymer is a pH sensitive polymer.

In one related aspect of the above embodiment, in the pharmaceutical composition the enteric polymer is selected from methacrylic acid copolymers, cellulose acetate phthalate, cellulose acetate succinate, polymethacrylic acid/acrylic acid copolymer, Kollicoat MAE 30DP, Eudragit, hydroxypropyl methylcellulose phthalate, polyvinyl acetate phthalate, hydroxyethyl ethyl cellulose phthalate, cellulose acetate tetrahydrophthalate, acrylic resin, or mixtures thereof.

In some aspect of the above embodiment, the composition of the present invention is a multiparticulate composition, comprising of plurality of discrete units.

In some aspects of the above embodiment, the composition of the present invention is in the form of a sachet, pouch or capsule.

In one aspect of the above embodiment, in the pharmaceutical composition of the present invention the nitrosamine drug substance-related impurities (NDSRIs) is nitrosoduloxetine.

In another aspect according to the above embodiment, in the pharmaceutical composition of the present invention the discrete unit is a granule, pellet, bead, particulate, spheroid or mini-tablet.

In some aspects according to the above embodiment, in the pharmaceutical composition the modified release coating is further coated with a protective layer or seal coating.

In one embodiment the pharmaceutical composition of duloxetine according to the present invention comprises duloxetine or a pharmaceutical acceptable salt thereof, and a stabilizing amount of at least one nitrite quencher, and one or more additional stabilizer selected from alkalizer and the likes, wherein the composition is characterized by nitrosamine drug substance-related impurities (NDSRIs) in the composition below about 1 ppm, preferably below about 0.9 ppm, more preferably below about 0.83 ppm.

In one embodiment, the pharmaceutical composition disclosed herein is for use in the treatment of a medical condition requiring the said amine drug.

In yet another embodiment, the disclosure provides a method of treating a medical condition requiring a pharmaceutical composition comprising the step of administering a therapeutically effective amount of a pharmaceutical composition, wherein said pharmaceutical composition comprises an amine drug with a nitrite quencher and has a reduced level of organic impurity and a nitrosamine impurity compared to a standard formulation of said drug.

In one aspect of the above embodiment, the reduced level of impurity in the said pharmaceutical composition is below about 1 ppm, preferably below about 0.9 ppm, more preferably below about 0.83 ppm upon storage at 40° C./75% RH for at least 6 months and at controlled room temperature condition of 25° C./60% RH for at least 12 months, 18 months or 24 months, and thereby enhancing the safety and efficacy of the treatment.

The present invention is illustrated below by reference to the following examples. However, one skilled in the art will appreciate that the specific methods and results discussed are merely illustrative of the invention, and not to be construed as limiting the invention, as many variations thereof are possible without departing from the spirit and scope of the invention.

### EXAMPLES

### Examples 1 and 2

| | | | |
|---|---|---|---|
| | | Ex-1 | Ex-2 |
| | | Quantity | Quantity |
| S.N. | Ingredients | (%/capsule) | (%/capsule) |

Drug Layering

| | | | |
|---|---|---|---|
| 1 | Sugar Spheres | 30-40% | 30-40% |
| 2 | Amine drug | 17-24% | 17-24% |
| 3 | Nitrite quencher | 0.01-10% | 0.01-10% |
| 4 | Talc | 0.01-10% | 0.01-10% |
| 5 | Hypromellose | 0.5-8% | — |
| 6 | Mannitol | — | 1-10% |
| 7 | Purified water | q.s. | q.s. |

Sub/Seal Coating

| | | | |
|---|---|---|---|
| 8 | Hypromellose | 0.5-8% | 3-12% |
| 9 | Sucrose | 1-10% | 3-10% |
| 10 | Talc | 4-16% | 1-8% |
| 11 | Purified water | q.s. | q.s. |

Enteric Coating

| | | | |
|---|---|---|---|
| 12 | Hypromellose Phthalate (HPMCP) | 8-20% | — |
| 13 | Methacrylic Acid and ethyl acrylate | — | 6-15% |
| | Copolymer Dispersion NF/Ph. Eur.[1:1] | | |
| | dispersion 30%] (Kollicoat MAE 30DP) | | |
| 14 | Triethyl Citrate | 0.1-4% | 0.5-5% |
| 15 | Talc | 0.3-5% | 0.4-8% |
| 16 | Sodium hydroxide | — | 0.001-0.008% |
| 17 | Purified water | q.s | q.s. |
| 18 | Acetone | q.s. | — |

Finishing Layer

| | | | |
|---|---|---|---|
| 19 | Hydroxypropylmethyl cellulose | 0.5-5% | — |
| 20 | Titanium Dioxide | 0.1-2% | — |
| 21 | Talc | 0.1-2% | — |
| 22 | Polyethylene Glycol 6000 USNF | 2-8% | — |
| 23 | Purified water | q.s. | — |

### Examples 3-8

| | | | | | | | |
|---|---|---|---|---|---|---|---|
| Ex-3 | Ex-4 | Ex-5 | Ex-6 | Ex-7 | Ex-8 | Ex-9 | Ex-10 |

| | | |
|---|---|---|
| S.N. | Ingredients | Quantity (%/capsule) |

Drug Layering

| | | | | | | | | | |
|---|---|---|---|---|---|---|---|---|---|
| 1 | Sugar Spheres | 37.7% | 32.4% | 32.5% | 39.9% | 35.1% | 33.3% | 38.9% | 39.4% |
| 2 | Duloxetine | 21.4% | 21.0% | 20.7% | 21.2% | 20.5% | 20.9% | 21.2% | 21.2% |
| | Hydrochloride USP | | | | | | | | |
| | equivalent to | | | | | | | | |
| | Duloxetine | | | | | | | | |
| 3 | Ascorbic acid | 0.4% | 0.31% | 0.5% | 0.3% | 0.5% | — | 0.3% | — |
| 4 | Butylated Hydroxy | — | — | — | — | — | — | 0.3% | — |
| | Anisole | | | | | | | | |
| 5 | Talc | 1.2% | 1.05% | 1.1% | 1.0% | 1.3% | 1.0% | 1.1 | 1.0% |
| 6 | Hypromellose | 2.3% | 2.09% | 2.0% | 2.11% | 2.5% | 2.0% | 2.1 | 2.1% |
| 7 | Mannitol | 4.0% | — | — | 4.2% | — | — | 4.2 | 4.2% |
| 8 | Purified water | q.s. | q.s. | q.s. | q.s. | q.s. | q.s. | q.s. | q.s. |

Sub/Seal Coating

| | | | | | | | | | |
|---|---|---|---|---|---|---|---|---|---|
| 9 | Hypromellose | 5.5% | 2.4% | 2.5% | 6.0% | 2.1% | 2.4% | 6.0% | 6.0% |
| 10 | Sucrose | 4.9% | 4.9% | 4.87% | 4.5% | 4.1% | 4.8% | 4.5% | 4.5% |
| 11 | Talc | 3.9% | 9.7% | 9.74% | 3.0% | 8.5% | 9.7% | 3.0% | 3.0% |
| 12 | Purified water | q.s. | q.s. | q.s. | q.s. | q.s. | q.s. | q.s. | q.s. |

Enteric Coating

| 13 | Hypromellose | — | 13.7% | 13.6% | — | 14.1% | 13.7% | — | — |
|---|---|---|---|---|---|---|---|---|---|
| | Phthalate (HPMCP) | | | | | | | | |
| 14 | Methacrylic Acid | 12.0% | — | — | 11.6% | — | — | 11.5% | 11.6% |
| | and ethyl acrylate | | | | | | | | |
| | Copolymer | | | | | | | | |
| | Dispersion | | | | | | | | |
| | NF/Ph. Eur.[1:1] | | | | | | | | |
| | dispersion 30%] | | | | | | | | |
| | (Kollicoat MAE | | | | | | | | |
| | 30DP) | | | | | | | | |
| 15 | Triethyl Citrate | 2.5% | 1.3% | 1.4% | 2.3% | 1.1% | 1.3% | 2.3% | 2.3% |
| 16 | Talc | 4.1% | 1.9% | 2.0% | 4.6% | 2.2% | 1.9% | 4.6% | 4.6% |
| 17 | Sodium hydroxide | 0.004% | — | — | 0.003% | — | — | 0.005% | 0.003% |
| 18 | Purified water | q.s. | q.s. | q.s. | q.s | q.s. | q.s. | q.s | q.s. |
| 19 | Acetone | q.s. | q.s. | q.s. | — | q.s. | q.s. | q.s | q.s. |

Finishing Layer

| | | | | | | | | | |
|---|---|---|---|---|---|---|---|---|---|
| 20 | Hydroxypropylmethyl | — | 1.8% | 1.8% | — | 1.5% | 1.8% | — | — |
| | cellulose | | | | | | | | |
| 21 | Titanium Dioxide | — | 0.9% | 0.9% | — | 0.5% | 0.9% | — | — |
| 22 | Talc | — | 0.9% | 0.9% | — | 1.2% | 0.9% | — | — |
| 23 | Polyethylene Glycol | — | 5.5% | 5.5% | — | 4.8% | 5.4% | — | — |
| | 6000 USNF | | | | | | | | |
| 24 | Purified water | — | q.s. | q.s. | — | q.s. | q.s. | — | — |

### Manufacturing Procedure of Example 1

1. Drug layering: Hydroxypropyl methylcellulose (HPMC/Hypromellose) was dissolved in purified water, amine drug, nitrite quencher and talc were suspended into the solution, and the solution was sprayed on sugar spheres to obtain drug-loaded pellets.
2. Sub coating: Hydroxypropylmethyl cellulose and sucrose were dissolved in purified water and talc was suspended into the solution, and the solution was sprayed on to the drug-loaded pellets of step 1.
3. Enteric coating: Hydroxypropylmethyl cellulose phthalate and triethyl citrate were dissolved in mixture of acetone and purified water, talc was suspended into the solution, and solution was sprayed on to sub-coated pellets of step 2.
4. Finishing layer: Hydroxypropylmethyl cellulose was dissolved in purified water; talc, titanium dioxide, polyethylene glycol (if present) were suspended into the solution, and the solution was sprayed on to the enteric loaded pellets of step 3.
5. The top-coated pellets of step 4 were filled in the capsule.

### Manufacturing Procedure of Example 2

1. Drug layering: Hydroxypropylmethyl cellulose (HPMC/Hypromellose) was dissolved in purified water, amine drug, nitrite quencher, mannitol and talc were suspended into the solution, and the solution was sprayed on sugar spheres to obtain drug-loaded pellets.
2. Sub coating: Hydroxypropylmethyl cellulose and sucrose were dissolved in purified water and talc was suspended into the solution, and the solution was sprayed on to the drug loaded pellets of step 1.
3. Enteric coating: Methacrylic Acid and ethyl acrylate Copolymer Dispersion NF/Ph.Eur.[1:1 dispersion 30%](Kollicoat MAE 30DP), Sodium hydroxide and triethyl citrate were disperse in purified water, talc was suspended into the solution, and solution was sprayed on to sub-coated pellets of step 2.
4. Enteric coated pellets of step 3 were filled in the capsule.

### Example 5: Dissolution Study

A comparative dissolution study was undertaken to see impact of including nitrite quencher in the composition, accordingly 4 batches were prepared with a formulation similar to
1. Example 1, as Batch 1A with nitrite quencher and batch 1B without nitrite quencher.
2. Example 2, as Batch 2A with nitrite quencher and batch 2B without nitrite quencher.
3. The dissolution study details of the all 4 batches are provided in Table 1-4 below:

TABLE 1
Dissolution details for batch (1B) prepared without ascorbic acid:

| | | 20 mg | | | 30 mg | | | 60 mg | | |
|---|---|---|---|---|---|---|---|---|---|---|
| STAGE | | Min. | Max. | Mean | Min. | Max. | Mean | Min. | Max. | Mean |
| Acid | 2 hour | 0 | 1 | 0 | 0 | 6 | 1 | 0 | 0 | 0 |
| Stage | | | | | | | | | | |
| Buffer | 15 min | 63 | 81 | 75 | 63 | 71 | 67 | 67 | 70 | 69 |
| Stage | 30 min | 65 | 92 | 85 | 74 | 87 | 82 | 79 | 85 | 81 |
| | 45 min | 86 | 97 | 92 | 86 | 94 | 90 | 81 | 89 | 86 |
| | 60 min | 87 | 99 | 94 | 86 | 97 | 92 | 86 | 92 | 90 |

Acid Stage: 0.1N HCl,
Buffer Stage: pH 6.8 Phosphate Buffer; USP-I Dissolution

TABLE 2
Dissolution details for batch (1A) prepared with ascorbic acid:

| | | 20 mg | | | 30 mg | | | 60 mg | | |
|---|---|---|---|---|---|---|---|---|---|---|
| STAGE | | Min | Max | Mean | Min | Max | Mean | Min | Max | Mean |
| Acid | 2 hour | 0 | 1 | 0 | 0 | 1 | 0 | 0 | 0 | 0 |
| Stage | | | | | | | | | | |
| Buffer | 15 min | 72 | 81 | 77 | 75 | 82 | 79 | 62 | 75 | 71 |
| Stage | 30 min | 84 | 92 | 88 | 86 | 94 | 90 | 75 | 87 | 83 |
| | 45 min | 90 | 97 | 93 | 91 | 97 | 94 | 81 | 93 | 89 |
| | 60 min | 92 | 98 | 95 | 93 | 100 | 97 | 89 | 96 | 92 |

Acid Stage: 0.1N HCl,
Buffer Stage: pH 6.8 Phosphate Buffer; USP-I Dissolution

TABLE 3
Dissolution details for batch (2B) prepared without ascorbic acid:

| | | 20 mg | | | 30 mg | | | 60 mg | | |
|---|---|---|---|---|---|---|---|---|---|---|
| STAGE | | Min. | Max. | Mean | Min. | Max. | Mean | Min. | Max. | Mean |
| Acid | 2 hour | 0 | 3 | 1 | 0 | 1 | 0 | 0 | 0 | 0 |
| Stage | | | | | | | | | | |
| Buffer | 15 min | 63 | 89 | 82 | 43 | 79 | 64 | 45 | 61 | 58 |
| Stage | 30 min | 92 | 102 | 98 | 84 | 95 | 90 | 83 | 91 | 86 |
| | 45 min | 94 | 104 | 99 | 90 | 96 | 93 | 87 | 93 | 91 |
| | 60 min | 95 | 103 | 100 | 92 | 97 | 94 | 87 | 95 | 93 |
| | 90 min | 96 | 105 | 100 | 92 | 97 | 95 | 89 | 94 | 93 |

Acid Stage: 0.1N HCl,
Buffer Stage: pH 6.8 Phosphate Buffer; USP-I Dissolution

TABLE 4
Dissolution details for batch (2A) prepared with ascorbic acid:

| | | 20 mg | | | 30 mg | | | 60 mg | | |
|---|---|---|---|---|---|---|---|---|---|---|
| STAGE | | Min | Max | Mean | Min | Max | Mean | Min | Max | Mean |
| Acid | 2 hour | 0 | 0 | 0 | 0 | 0 | 0 | 0 | 0 | 0 |
| Stage | | | | | | | | | | |
| Buffer | 15 min | 58 | 77 | 66 | 27 | 73 | 59 | 49 | 66 | 59 |
| Stage | 30 min | 90 | 99 | 102 | 79 | 97 | 92 | 95 | 100 | 97 |
| | 45 min | 91 | 102 | 98 | 87 | 100 | 96 | 98 | 103 | 100 |
| | 60 min | 93 | 104 | 99 | 95 | 101 | 99 | 101 | 107 | 103 |
| | 90 min | 95 | 105 | 100 | 95 | 101 | 99 | 98 | 106 | 102 |

Acid Stage: 0.1N HCl,
Buffer Stage: pH 6.8 Phosphate Buffer; USP-I Dissolution

For dissolution study 12 samples from each of the batches 1A, 1B, 2A and 2 B were run, and mean, minimum and maximum values for each batch is provided in the above tables. Nitrite quencher was incorporated in to the composition for e.g. 1 mg of ascorbic acid (60 mg strength), similarly other strengths were prepared and tested for all batches.

### Observation:

There was no significant difference in bioequivalence data between the two batches 1A and 1B, 2A and 2B respectively. So, the composition with or without nitrite quencher were bioequivalent. Hence, with addition of ascorbic acid, pharmacokinetic profile is not altered. Also, comparative dissolution profile (CDP) data of:
1. batch 2A (with ascorbic acid) and 2B (without ascorbic acid); and
2. batch 1A (with ascorbic acid) and 1B (without ascorbic acid),
3. a dissolution similarity was established respectively.

### Example 6: Stability Study

Stability study was undertaken for final compositions of batch 2A (with nitrite quencher) with all strengths, for example 20 mg, 30 mg, 40 mg and 60 mg. Stability Data (for 20 mg and 60 mg) with respect to dissolution, water content, related substance, content of NDSRI and assay is provided in below tables:

TABLE 5
Stability details for batch (2A) 20 mg strength prepared with ascorbic acid:

| | | | Accelerated Stability | | | Long Term Stability | | | | | | |
|---|---|---|---|---|---|---|---|---|---|---|---|---|
| | Specification | | (20 mg) | | | (20 mg) | | | | | Photostability | |
| Parameter | Stage | | 0 M | 3 M | 6 M | 0 M | 3 M | 6 M | 9 M | 12 M | DE | DU |
| Dissolution | Acid Stage | Min | 0 | 0 | 0 | 0 | 0 | 0 | 0 | 0 | 0 | 0 |
| (Release in | | Max | 0 | 0 | 2 | 0 | 0 | 2 | 0 | 0 | 0 | 0 |
| %) | | Mean | 0 | 0 | 0 | 0 | 0 | 0 | 0 | 0 | 0 | 0 |
| | Buffer | Min | 97 | 94 | 99 | 97 | 91 | 96 | 90 | 97 | 95 | 94 |
| | Stage | Max | 102 | 105 | 106 | 102 | 98 | 105 | 105 | 104 | 100 | 98 |
| | | Mean | 99 | 99 | 103 | 99 | 95 | 98 | 96 | 100 | 97 | 96 |
| Water | NMT 4.0% (in %) | | 1.73 | 1.51 | 1.49 | 1.73 | 1.51 | 1.30 | 1.73 | 1.88 | 1.31 | 1.26 |
| content (KF) | | | | | | | | | | | | |
| Related | 1-Naphthol | NMT | BQL | 0.056 | 0.085 | BQL | 0.011 | BQL | BQL | BQL | 0.013 | 0.014 |
| Substance | | 0.2% | | | | | | | | | | |
| | Unk. Imp. | NMT | 0.038 | 0.051 | 0.053 | 0.038 | 0.043 | BQL | BQL | BQL | 0.184 | 0.162 |
| | | 0.2% | | | | | | | | | | |
| | Total Imp. | NMT | 0.038 | 0.107 | 0.138 | 0.038 | 0.054 | BQL | BQL | BQL | 0.197 | 0.176 |
| | | 0.4% | | | | | | | | | | |
| Content of | NMT 0.833 ppm | | 0.35 | 0.498 | 0.274 | 0.35 | 0.514 | 0.306 | 0.300 | 0.557 | — | — |
| NDSRI | | | | | | | | | | | | |

| | | | | | | | | | | | |
|---|---|---|---|---|---|---|---|---|---|---|---|
| Assay | (Content of duloxetine | 103.1 | 100.5 | 99.6 | 103.1 | 102.9 | 99.1 | 99.7 | 100.0 | 101.1 | 102.0 |
| | between 90%-110%) | | | | | | | | | | |

Accelerated stability: 40° C. ± 2° C. and 75% ± 5% RH; Long term stability: 25° C. ± 2° C. and 60% ± 5% RH; Pack: HDPE Container.

TABLE 6
Stability details for batch (2A) 60 mg strength prepared with ascorbic acid:

| | | | Accelerated Stability | | | Long Term Stability | | | | | | |
|---|---|---|---|---|---|---|---|---|---|---|---|---|
| | Specification | | (60 mg) | | | (60 mg) | | | | | Photostability | |
| Parameter | Stage | | 0 M | 3 M | 6 M | 0 M | 3 M | 6 M | 9 M | 12 M | DE | DU |
| Dissolution | Acid Stage | Min | 0 | 0 | 0 | 0 | 0 | 0 | 0 | 0 | 0 | 0 |
| (Release in | | Max | 0 | 1 | 0 | 0 | 0 | 0 | 0 | 1 | 1 | 1 |
| %) | | Mean | 0 | 0 | 0 | 0 | 0 | 0 | 0 | 0 | 0 | 0 |
| | Buffer | Min | 93 | 96 | 94 | 93 | 93 | 94 | 91 | 94 | 94 | 94 |
| | Stage | Max | 103 | 102 | 97 | 103 | 99 | 98 | 96 | 98 | 99 | 99 |
| | | Mean | 98 | 99 | 96 | 98 | 96 | 96 | 93 | 96 | 96 | 98 |
| Water | NMT 4.0% (in %) | | 1.51 | 1.47 | 1.43 | 1.52 | 1.34 | 1.26 | 1.41 | 1.71 | 1.62 | 1.61 |
| content (KF) | | | | | | | | | | | | |
| Related | 1-Naphthol | NMT | BQL | 0.066 | 0.114 | BQL | 0.018 | BQL | BQL | 0.025 | 0.013 | 0.014 |
| Substance | | 0.2% | | | | | | | | | | |
| | Unk. Imp. | NMT | 0.038 | 0.052 | 0.060 | 0.038 | 0.054 | BQL | BQL | BQL | BQL | BQL |
| | | 0.2% | | | | | | | | | | |
| | Total Imp. | NMT | 0.038 | 0.118 | 0.222 | 0.038 | 0.072 | BQL | BQL | 0.025 | 0.013 | 0.014 |
| | | 0.4% | | | | | | | | | | |
| Content of | NMT 0.833 ppm | | 0.36 | 0.504 | 0.316 | 0.36 | 0.474 | 0.334 | 0.25 | 0.408 | — | — |
| NDSRI | | | | | | | | | | | | |

| | | | | | | | | | | | |
|---|---|---|---|---|---|---|---|---|---|---|---|
| Assay | (Content of duloxetine | 101.7 | 101.0 | 100.2 | 101.7 | 102.9 | 98.9 | 96.9 | 102.1 | 102.7 | 102.4 |
| | between 90%-110%) | | | | | | | | | | |

Accelerated stability: 40° C. ± 2° C. and 75% ± 5% RH; Long term stability: 25° C. ± 2° C. and 60% ± 5% RH; Pack: HDPE Container.

### Observation:

There was no significant difference in bioequivalence data in the accelerated stability study at least up to 6 months and in the long-term stability study at least up to 12 months. The composition with nitrite quencher were found to be within the specification for all test parameters including assay:
1. Water content not more than (NMT) 4%.
2. Related substance:
   1-Naphthol: not more than (NMT) 0.2%.
   Unknown Impurity: not more than (NMT) 0.2%.
   Total Impurity: not more than (NMT) 0.4%.
3. Content of NDSR: not more than (NMT) 0.833 PPM.
4. Assay: content of duloxetine between 90%-110%.

Further stability study was done for sample of batch A (with nitrite quencher) with all strengths 20 mg, 30 mg, 40 mg and 60 mg. Stability Data with respect to dissolution, water content, organic impurity and content of NDSRI and assay is provided in below tables:

TABLE 7
Stability details for batch (1A) prepared with ascorbic acid:

| | Specification | | Strength | | | | | | | |
|---|---|---|---|---|---|---|---|---|---|---|
| Parameter | Stage - 12 M CRT | | 20 | | 30 | | 40 | | 60 | |
| Dissolution | Acid Stage 2 hr | NMT 10% | 1 | (1-2) | 0 | (0-0) | 1 | (0-1) | 1 | (1-1) |
| (By HPLC) | Buffer Stage 45 | >=85% | 97 | (92-98) | 94 | (91-97) | 93 | (89-96) | 91 | (86-93) |
| | min. | Q = 80 | | | | | | | | |
| Water | NMT 3.0% | | 1.2 | | 1.1 | | 1.1 | | 1.1 | |
| content (KF) | | | | | | | | | | |
| Organic | 1-Naphthol | NMT 0.2% | BQL | (0.03) | BQL | (0.03) | BQL | (0.03) | 0.06 | |
| Impurity | Duloxetine 4- | NMT 0.2% | BQL | (0.016) | BQL | (0.017) | BQL | (0.015) | BQL | (0.018) |
| (in % w/w) | napthyl isomer | | | | | | | | | |
| | Duloxetine Beta | NMT 0.2% | BQL | (0.015) | BQL | (0.018) | BQL | (0.017) | BQL | (0.029) |

| | naphthol-1-yl | | | | | |
|---|---|---|---|---|---|---|
| | isomer. | | | | | |
| | Any individual | NMT 0.2% | BQL | BQL | BQL | BQL |
| | unspecified deg. | | | | | |
| | product | | | | | |
| | Total impurity | NMT 0.4% | BQL | BQL | BQL | 0.06 |
| Content of | NMT 0.833 ppm | | 0.159 | 0.161 | 0.129 | 0.176 |
| NDSRI | | | | | | |

| | | | | | |
|---|---|---|---|---|---|
| Assay | (Content of duloxetine | 100.8 | 101.9 | 101.9 | 100.7 |
| | between 90%-110%) | | | | |

The stability study depicted that by incorporation of nitrite quencher there was significant improvement in stability of the formulation. The key impurities i.e. the organic impurities 1-Naphthol, duloxetine 4-napthyl isomer, Duloxetine Beta naphthol-1-yl isomer, any individual unspecified drug product, total impurity and also the NDSRI impurity were both in control and were found to be within the impurity specification in the finished product prepared in the present disclosure.

### Examples 7: Comparative Stability Study for Marketed Product, Batches without Nitrite Quencher and Batches with Nitrite Quencher

| | | | | Date of | Nitrosoduloxetine |
|---|---|---|---|---|---|
| Batch no. | Strength | Date of Mfg. | Date of Exp. | Analysis | impurity (ppm) |
| CYMBALTA | 60 mg | | January 2023 | Dec. 10, | 1.390 |
| D199780A | | | | 2022 | |
| CYMBALTA | | | January 2023 | Dec. 10, | 1.319 |
| D317314A | | | | 2022 | |
| Batch 1 B | 60 mg | February 2021 | January 2023 | Aug. 24, | 5.60 |
| (Without Ascorbic | | | | 2021 | |
| Acid) | | | | | |
| Batch 1 B | | March 2021 | February 2023 | Aug. 24, | 3.34 |
| (Without Ascorbic | | | | 2021 | |
| Acid) | | | | | |
| Batch 1 B | | April 2021 | March 2023 | Aug. 24, | 1.35 |
| (Without Ascorbic | | | | 2021 | |
| Acid) | | | | | |
| Batch 1 A | 20 mg | April 2022 | March 2024 | — | 0.1339 |
| (With Ascorbic | 30 mg | April 2022 | | | 0.1396 |
| Acid) | 40 mg | April 2022 | | | 0.1545 |
| | 60 mg | April 2022 | | | 0.1546 |

### Observation:

There was significant difference in nitrosamine impurity levels when compared with existing brand formulation and other formulations prepared without nitrite quencher. The composition with nitrite quencher were found to be within the specification for Content of NDSRI i.e. not more than (NMT) 0.833 PPM.

The stability study depicted that by incorporation of nitrite quencher there was significant improvement in stability of the formulation.

1. A pharmaceutical composition comprising a plurality of discrete units, wherein each unit comprises:
a drug layered pellet comprising an inert core coated with a drug layer, wherein the drug layer comprises duloxetine hydrochloride and an effective amount of nitrile quencher to reduce nitrosoduloxetine in the composition, wherein the nitrile quencher comprises ascorbic acid, cysteine, histidine, glycine, arginine, a salt thereof, or a combination thereof;
a sub coating comprising a film-forming polymer selected from hydroxypropyl methylcellulose, ethyl cellulose, methylcellulose, hydroxyethyl cellulose, hydroxypropyl cellulose, sodium carboxymethyl cellulose, cellulose acetate, hydroxypropyl methylcellulose phthalate, cellulose acetate trimellitate, a methacrylic acid copolymer, polyvinylpyrrolidone, polyvinyl alcohol, polyethylene glycol, and combinations thereof, wherein the sub coating covers the drug-layered pellet; and
an enteric coating comprising an enteric polymer selected from a methacrylic acid copolymer, cellulose acetate phthalate, cellulose acetate succinate, polymethacrylic acid/acrylic acid copolymer, hydroxypropyl methylcellulose phthalate, polyvinyl acetate phthalate, hydroxyethyl ethyl cellulose phthalate, cellulose acetate tetrahydrophthalate, acrylic resin, and combinations thereof, wherein the enteric coating covers the sub coating;
wherein the composition comprises less than about 1 PPM nitrosoduloxetine following storage for six months at 40° C. and 75% relative humidity (RH).

2. The composition of claim 1, comprising less than 0.2% w/w 1-naphthol following storage for six months at 40° C. and 75% RH.

3. The composition of claim 1, wherein the nitrile quencher comprises ascorbic acid.

4. The composition of claim 1, wherein the film-forming polymer comprises hydroxypropyl methylcellulose, and wherein the sub coating further comprises sucrose and/or talc.

5. The composition of claim 1, wherein the enteric polymer comprises hydroxypropyl methylcellulose phthalate or a methacrylic acid copolymer, and wherein the enteric coating further comprises one or more of triethyl citrate, talc, and sodium hydroxide.

6. The composition of claim 1, wherein the cushioning agent comprises polyethylene glycol, and wherein the finishing layer further comprises one or more of hydroxypropyl methylcellulose, titanium dioxide, and talc.

7. The composition of claim 1, wherein the duloxetine hydrochloride is about 18% to about 25% by weight of the composition.

8. The composition of claim 1, wherein the nitrile quencher is about 0.01% to about 10% by weight of the composition.

9. The composition of claim 1, wherein the hydroxypropyl methylcellulose is about 3% to about 12% by weight of the composition.

10. The composition of claim 1, wherein the enteric polymer is about 55% to about 98% by weight of the enteric coating, and wherein the enteric coating is about 15% to about 30% by weight of each unit without the enteric coating.

11. The composition of claim 1, further comprising a finishing layer comprising a cushioning agent, wherein the finishing layer covers the enteric coating.

12. The composition of claim 1, wherein the cushioning agent is about 2% to about 20% by weight of the composition.

13. The composition of claim 1, wherein the composition comprises about 20 mg, about 30 mg, about 40 mg, about 50 mg, about 60 mg, about 70 mg, about 80 mg, about 90 mg, about 100 mg, about 110 mg, or about 120 mg duloxetine hydrochloride.

14. The composition of claim 1, wherein the plurality of discrete units are in a form of pellets, beads, particles, granules, mini-tablets, or combinations thereof.

15. The composition of claim 1, wherein the plurality of discrete units have an average diameter of about 500 μm to about 1800 μm.

16. The composition of claim 1, wherein the composition is in a form of a tablet, a capsule, a sachet, or granules.

17. A multiparticulate sprinkle dosage form comprising a plurality of discrete units, wherein each unit comprises:
a drug layered pellet comprising a sugar sphere coated with a drug layer, wherein the drug layer comprises duloxetine hydrochloride and ascorbic acid;
a sub coating comprising: hydroxypropyl methylcellulose; sucrose; and talc, wherein the sub coating covers the drug-layered pellet;
an enteric coating comprising: an enteric polymer selected from hydroxypropyl methylcellulose phthalate and a methacrylic acid copolymer; triethyl citrate; talc; and sodium hydroxide, wherein the enteric coating covers the sub coating; and
a finishing layer comprising polyethylene glycol; hydroxypropyl methylcellulose; titanium dioxide; and talc, wherein the finishing layer covers the enteric coating.

18. The dosage form of claim 17, comprising less than about 1 PPM nitrosoduloxetine and/or less than 0.2% w/w 1-naphthol following storage for six months at 40° C. and 75% relative humidity (RH).

19. The dosage form of claim 17, wherein the duloxetine hydrochloride is about 18% to about 25% by weight of the dosage form, and the ascorbic acid is about 0.01% to about 10% by weight of the dosage form.

20. The dosage form of claim 17, wherein
the drug layered pellet comprises:
about 30% to about 40% w/w sugar sphere;
about 17% to about 24% w/w duloxetine hydrochloride;
about 0.01% to about 10% w/w ascorbic acid;
about 0.01% to about 10% w/w talc; and
about 0.5% to about 8% w/w hydroxypropyl methylcellulose;
the sub coating comprises:
about 0.5% to about 8% w/w hydroxypropyl methylcellulose;
about 1% to about 10% w/w sucrose; and
about 4% to about 16% talc;
the enteric coating comprises:
about 8% to about 20% w/w hydroxypropyl methylcellulose phthalate;
about 0.1% to about 4% w/w triethyl acetate; and
about 0.3% to about 5% talc; and
the finishing layer comprises:
about 0.5% to about 5% w/w hydroxypropyl methylcellulose;
about 0.1% to about 2% w/w titanium dioxide;
about 0.1% to about 2% w/w talc; and
about 2% to about 8% w/w polyethylene glycol.

21. A method of treating a subject in need of duloxetine therapy, comprising administering to the subject a pharmaceutical composition comprising:
a drug layered pellet comprising an inert core coated with a drug layer, wherein the drug layer comprises duloxetine hydrochloride and an effective amount of nitrile quencher to reduce nitrosoduloxetine in the composition, wherein the nitrile quencher comprises ascorbic acid, cysteine, histidine, glycine, arginine, a salt thereof, or a combination thereof;
a sub coating comprising a film-forming polymer selected from hydroxypropyl methylcellulose, ethyl cellulose, methylcellulose, hydroxyethyl cellulose, hydroxypropyl cellulose, sodium carboxymethyl cellulose, cellulose acetate, hydroxypropyl methylcellulose phthalate, cellulose acetate trimellitate, a methacrylic acid copolymer, polyvinylpyrrolidone, polyvinyl alcohol, polyethylene glycol, and combinations thereof, wherein the sub coating covers the drug-layered pellet;
an enteric coating comprising an enteric polymer selected from a methacrylic acid copolymer, cellulose acetate phthalate, cellulose acetate succinate, polymethacrylic acid/acrylic acid copolymer, hydroxypropyl methylcellulose phthalate, polyvinyl acetate phthalate, hydroxyethyl ethyl cellulose phthalate, cellulose acetate tetrahydrophthalate, acrylic resin, and combinations thereof, wherein the enteric coating covers the sub coating; and
a finishing layer comprising a cushioning agent, wherein the finishing layer covers the enteric coating,
wherein the composition comprises less than about 1 PPM nitrosoduloxetine and/or less than 0.2% w/w 1-naphthol following storage for six months at 40° C. and 75% relative humidity (RH).

22. The method of claim 21, wherein the pharmaceutical composition is administered to the subject as a sprinkle dosage form, as an intact capsule, or via a feeding tube.
